SEQUENCE LISTING (46 sequences)

SEQ ID NO: 1 - DNA, 20 bp, Artificial Sequence
  primer
tcagcctcttctccttcctg

SEQ ID NO: 2 - DNA, 20 bp, Artificial Sequence
  primer
gccagagggctgattagaga

SEQ ID NO: 3 - DNA, 20 bp, Artificial Sequence
  primer
aagcccttgctgtagtggtg

SEQ ID NO: 4 - DNA, 20 bp, Artificial Sequence
  primer
gaagctgatggccctaaaca

SEQ ID NO: 5 - DNA, 20 bp, Artificial Sequence
  primer
tgagccacactgaaaaggaa

SEQ ID NO: 6 - DNA, 21 bp, Artificial Sequence
  primer
ggtgaatttctgctccattca

SEQ ID NO: 7 - DNA, 20 bp, Artificial Sequence
  primer
cagcgcttgtgatcttcatt

SEQ ID NO: 8 - DNA, 20 bp, Artificial Sequence
  primer
tacccctgctcctggttttt

SEQ ID NO: 9 - DNA, 20 bp, Artificial Sequence
  primer
cagcttcgtgcctttcataa

SEQ ID NO: 10 - DNA, 19 bp, Artificial Sequence
  primer
ctcccactgcacctgtagc

SEQ ID NO: 11 - DNA, 20 bp, Artificial Sequence
  primer
gtcgatgtcacatgcagctt

SEQ ID NO: 12 - DNA, 20 bp, Artificial Sequence
  primer
gaagaaaaccccttgtgctg

SEQ ID NO: 13 - DNA, 20 bp, Artificial Sequence
  primer
ctgccatccatccagaactc

SEQ ID NO: 14 - DNA, 20 bp, Artificial Sequence
  primer
agcactgcctcttcatggtc

SEQ ID NO: 15 - DNA, 20 bp, Artificial Sequence
  primer
cctatggccctcattctcac

SEQ ID NO: 16 - DNA, 20 bp, Artificial Sequence
  primer
ctcatcctgctgggtctgag

SEQ ID NO: 17 - DNA, 20 bp, Artificial Sequence
  primer
gtggattgtcttgtggagca

SEQ ID NO: 18 - DNA, 20 bp, Artificial Sequence
  primer
ttgtggtgagctgaaaggtg

SEQ ID NO: 19 - DNA, 22 bp, Artificial Sequence
  primer
tttctccagtgtagccatcctt

SEQ ID NO: 20 - DNA, 20 bp, Artificial Sequence
  primer
aggagcaggaatcattgacg

SEQ ID NO: 21 - DNA, 20 bp, Artificial Sequence
  primer
tctcccacaggaggtttctg

SEQ ID NO: 22 - DNA, 20 bp, Artificial Sequence
  primer
acagagttccaggccatcaa

SEQ ID NO: 23 - DNA, 20 bp, Artificial Sequence
  primer
tgtggggtggagatctcagt

SEQ ID NO: 24 - DNA, 20 bp, Artificial Sequence
  primer
tctccttcctggacacatga

SEQ ID NO: 25 - DNA, 20 bp, Artificial Sequence
  primer
gtcaggggtggttattgcat

SEQ ID NO: 26 - DNA, 20 bp, Artificial Sequence
  primer
agtgaggaacaagccagagc

SEQ ID NO: 27 - DNA, 22 bp, Artificial Sequence
  primer
tcaaactcactcatggctttgt

SEQ ID NO: 28 - DNA, 20 bp, Artificial Sequence
  primer
gctgtcatcgatttcttccc

SEQ ID NO: 29 - DNA, 20 bp, Artificial Sequence
  primer
gtggaatctgccaggaggta

SEQ ID NO: 30 - DNA, 19 bp, Artificial Sequence
  primer
tccttgtcctcgtctgcac

SEQ ID NO: 31 - DNA, 21 bp, Artificial Sequence
  primer
ttcaccactcccaaaacctgc

SEQ ID NO: 32 - DNA, 22 bp, Artificial Sequence
  primer
gaggccaggcaactcccattag

SEQ ID NO: 33 - DNA, 22 bp, Artificial Sequence
  primer
agacctcagtggtgtagcagag

SEQ ID NO: 34 - DNA, 22 bp, Artificial Sequence
  primer
tgatgaccagcggttcaggatc

SEQ ID NO: 35 - DNA, 20 bp, Artificial Sequence
  primer
ggtctgggccatagaactga

SEQ ID NO: 36 - DNA, 20 bp, Artificial Sequence
  primer
cagcctcttctcattcctgc

SEQ ID NO: 37 - DNA, 20 bp, Artificial Sequence
  primer
taggcaggtttgatctccgt

SEQ ID NO: 38 - DNA, 21 bp, Artificial Sequence
  primer
cgatccactacaaatccctca

SEQ ID NO: 39 - DNA, 20 bp, Artificial Sequence
  primer
tttttccagcagaccagctt

SEQ ID NO: 40 - DNA, 20 bp, Artificial Sequence
  primer
agagattatcggagcgcctt

SEQ ID NO: 41 - DNA, 20 bp, Artificial Sequence
  primer
agacaccttggtcttggagc

SEQ ID NO: 42 - DNA, 20 bp, Artificial Sequence
  primer
tttgaattccctgggtgaga

SEQ ID NO: 43 - DNA, 20 bp, Artificial Sequence
  primer
accagctcaccaacttcctg

SEQ ID NO: 44 - DNA, 20 bp, Artificial Sequence
  primer
tgcttctggggacttttctg

SEQ ID NO: 45 - DNA, 20 bp, Artificial Sequence
  primer
agcggagtcggtacttgaga

SEQ ID NO: 46 - DNA, 20 bp, Artificial Sequence
  primer
tcctagcctttgtggctgag

(19) United States
(12) Patent Application Publication
(10) Pub. No.: US 2018/0194838 A1
(43) Pub. Date: Jul. 12, 2018

(54) METHOD FOR TREATING CANCER METASTASIS AND COMPOSITION THEREOF

(71) Applicant: NATIONAL YANG-MING UNIVERSITY, Taipei City (TW)

(72) Inventors: Muh-Hwa YANG, Taipei City (TW); Chih-Chan LEE, New Taipei City (TW)

(73) Assignee: NATIONAL YANG-MING UNIVERSITY, Taipei City (TW)

(21) Appl. No.: 15/866,001

(22) Filed: Jan. 9, 2018

Related U.S. Application Data
(60) Provisional application No. 62/444,535, filed on Jan. 10, 2017.

(51) Int. Cl. C07K 16/24; A61P 35/04; C12N 15/113; A61K 39/395; A61K 9/00; A61K 45/06

(52) U.S. Cl. CPC C07K 16/244; A61P 35/04; C12N 15/1138; C07K 16/243; A61K 39/3955; A61K 2039/507; A61K 9/0053; A61K 9/0073; A61K 9/0014; A61K 45/06; A61K 9/0019

(57) ABSTRACT

The present invention is related to a method for treating cancer metastasis and composition thereof. By using an IL-35 antagonist, cancer metastasis can be effectively treated so that an increased cancer fee and overall survival can be achieved.

### CROSS REFERENCE TO RELATED APPLICATIONS

This non-provisional application claims the benefit under 35 U.S.C. § 119(e) to U.S. Provisional Application No. 62/444,535, filed on Jan. 10, 2017, which is hereby expressly incorporated by reference into the present application.

### TECHNICAL FIELD

The present invention relates to cancer therapy, especially to cancer therapy for inhibiting, reducing, and/or preventing cancer metastasis.

### DESCRIPTION OF RELATED ART

Cancer is a major cause of death in the world. Although primary treatments of cancer (surgery, radiation therapy, and chemotherapy) are beneficial and lead to increased cancer free and overall survival, there is a continuous relapse rate that leads to a substantial proportion of cancer patients developing recurrent and/or metastatic cancer. Most people who die of cancer do not die from their primary tumor; they die from metastatic disease. When patients have surgery, surgeons don't know if there are other, smaller lesions elsewhere in the body. There remains a need in the art for therapeutic agents that effectively prevent cancer metastasis and recurrence.

### SUMMARY

One of the objectives of the present invention is to increase cancer free and overall survival of cancer patients by treating cancer metastasis. Another objective of the present invention is to provide a composition or a kit thereof to assist in primary cancer treatment so that the cancer metastasis is treated.

In order to achieve the aforesaid objectives, the present invention provides a method for treating cancer metastasis, comprising administering a subject in need a therapeutically effective amount of an interleukin-35 (IL-35) antagonist.

The present invention also provides a use of IL-35 antagonist in preparing a pharmaceutical composition for treating cancer metastasis; wherein said pharmaceutical composition comprises a therapeutically effective amount of said IL-35 antagonist and a pharmaceutically acceptable carrier.

The present invention then provides a pharmaceutical composition for treating cancer metastasis of a subject having had a primary cancer treatment, comprising: a therapeutically effective amount of an IL-35 antagonist; and a pharmaceutically acceptable carrier.

The present invention more provides a kit for treating cancer metastasis of a subject having had a primary cancer treatment, comprising: a first container comprising an IL-35 antagonist; and a second container comprising a CSF1R antagonist.

The present invention further provides a method for treating cancer, comprising (a) administering a subject in need with a primary cancer treatment; and (b) administering said subject a therapeutically effective amount of an IL-35 antagonist and/or a therapeutically effective amount of a CSF1R antagonist.

In light of the foregoing, the present invention provides a use of IL-35 antagonist for treating cancer metastasis. Accordingly, the present invention provides a method, pharmaceutical composition, and kit for treating cancer metastasis by using an IL-35 antagonist. The present invention successfully achieves increased cancer free and overall survival and is valuable for cancer treating regimen.

### BRIEF DESCRIPTION OF THE DRAWINGS

The patent or application file contains at least one color drawing. Copies of this patent or patent application publication with color drawing will be provided by the USPTO upon request and payment of the necessary fee.

FIG. 1A shows the result of RT-qPCR for analyzing the expression of M1 (Nos2, Tnf, IL15, Cxcl9, and Cxcl10) and M2 markers (Arg1, Mrc1, Il10, Chil3, and Ccl17) in CD11b⁺F4/80⁺ TAMs isolated from the primary tumors (pTAMs; p) and metastatic lungs (mTAMs; m) 5 weeks after inoculation of 4T1 cells. The data is normalized to bone marrow-derived macrophages (BMDM) from healthy mice. n=3. Data represent mean±S.E.M. ***p<0.001. n.s.=non-significance.

FIG. 1B shows the results of RT-qPCR for analyzing the expression of M2 markers (Arg1, Mrc1, Il10, and Chil3) in CD11b⁺F4/80⁺Mrc1⁺ cells from the primary tumors (pTAMs; p) and metastatic lungs (mTAMs; m) 5 weeks after inoculation of 4T1 cells. The data is normalized to BMDM (n=3) from healthy mice. Data represent mean±S.E.M. **p<0.01; ***p<0.001.

FIG. 1C shows the results of RT-qPCR for analyzing the expression of M1 (TNFA, IL6, IL1B) and M2 markers (IL10, CD163, CCL18) in CD14⁺ TAMs from primary (n=11) and metastatic human cancers (n=12). The data is normalized to peripheral blood monocytes-derived macrophages (PMMs) (n=5). Data represent mean±S.E.M. The p value is show in panel. n.s.=non-significance.

FIG. 2A shows the results of the MTT assay for analyzing the viability of 4T1 cells under PBS or liposomal clodronate treatment (50,100, and 200 μg/ml for 24 hr). n=2.

FIG. 2B illustrates the schema of pulmonary macrophage depletion in 4T1 orthotopic experiments through intratracheal injection of liposomal clodronate.

FIG. 2C shows the quantification of F4/80⁺ macrophages in lungs of mice receiving intratracheal liposomal clodronate or vehicle control (PBS). The data is presented as the relative fold change of the F4/80⁺ population in 6 representative fields. Data represent mean±S.E.M. **p<0.01. (n=5 for each group).

FIG. 2D concludes the effects of macrophages depletion on metastasis. Upper: photos of primary tumors (left) and representative photos of lungs (right) of mice receiving intratracheal liposomal clodronate or control PBS. Red arrows indicate the nodules in metastatic lung. Lower: quantification of the results. Scale bar, 1 cm. Data represent mean±S.E.M. **p<0.01. (n=5 for each group).

FIG. 2E shows the quantification of metastatic lung nodules of the Ly6c⁻ TAM co-injection experiment (Experiment 2) 2 weeks after tumor cells injection. Data represent mean t S.E.M. ***p<0.001. (n=7 for each group).

FIG. 2F shows the results of the Mrc1⁺ TAM co-injection experiment (Experiment 2). Upper: representative photos of lungs from mice 2 weeks after injection of the 4T1 cells with/without Mrc1⁺ TAMs. Lower: quantification of metastatic lung nodules. Data represent mean±S.E.M. **p<0.01. (n=6 for each group).

FIG. 3A illustrates the procedures of Experiment 3 of the specification. (MΦ: macrophages)

FIG. 3B exhibits the results of the Western blot of E-cadherin and N-cadherin in 4T1 cells treated with the indicated conditioned media for 48 hr. BMDM, bone marrow derived macrophages; pTAM, CD11b⁺F4/80⁺ primary TAM; mTAM, CD11b⁺F4/80⁺ metastatic TAMs.

FIG. 3C displays phase-contrast images, which showed the morphology of A549 and 4T1 cells incubated with different macrophage conditioned media for 48 hr. Scale bar, 50 μm.

FIG. 3D shows the results of the transwell migration assay for analyzing the migratory ability of A549 cells incubated with different macrophage conditioned media for 48 hr. n=2. Data represent mean±S.E.M. *p<0.05.

FIG. 3E exhibits the results of the surface expression of M1 (HLA-DR) and M2 (MR and CD163) markers in polarized macrophages from human CD14⁺ monocytes by flow cytometry analysis.

FIG. 3F shows the results of the RT-qPCR for analyzing the expression of M1 (IL1B, IL6, and IFNG) and M2 (MRC1, CD163, and CCL18) markers in polarized macrophages from human CD14⁺ monocytes to confirm the successful polarization. n=2. Data represent mean±S.E.M. **p<0.01; ***p<0.001.

FIG. 3G shows the results of the endothelial cell tube formation assay. Upper: representative image of HUVEC organization. Scale bar, 50 μm. Lower: quantification of the tube formation by measuring the branch point number when co-culture with M0, M1, and M2 conditioned media for 12 hours. n−3. Scale bar, 50 μm. (M0 CM: resting macrophages conditioned media; M1 CM: M1 macrophages conditioned media; M2 CM: M2 macrophages conditioned media). Data represent mean±S.E.M. *p<0.05.

FIG. 3H displays the Western blot of E-cadherin, N-cadherin, vimentin and γ-catenin in OECM1, 4T1 and A549 cells upon treatment of the indicated conditioned media for 48 hr.

FIG. 3I shows the immunofluorescent staining of E-cadherin, N-cadherin, and vimentin in 4T1, OECM1, and A549 cells upon treatment of the indicated conditioned media for 48 hr. Scale bar, 100 μm.

FIG. 3J shows the results of the transendothelial migration assay in Experiment 3. Upper: transendothelial migration assay of A549 and OECM1 cells upon indicated conditioned media treatment. Scale bar, 100 μm. Lower: quantification of cancer cell migration. n=3. The data is presented as the relative fold change of migrating cell number. Data represent mean±S.E.M. **p<0.01; ***p<0.001.

FIG. 3K displays the hematoxylin & eosin stain of the tumor samples harvested from the orthotopic SAS xenograft mouse model. The SAS cells are treated with the indicated conditioned media for 48 hr before inoculation. Scale bar, 200 μm. (n=5 for each group).

FIG. 3L shows the in vivo metastatic colonization ability of M1 CM or M2 CM treated A549 cells. Upper: representative photos of the lungs from mice receiving tail vein injection of A549 cells pretreated with M1 or M2 CM or control media. Lower: quantification of metastatic lung nodules 8 weeks after tumor cells injection (n=6 for each group).

FIG. 4A exhibits the results of RT-qPCR for analyzing the expression of Il12a and Ebi3 in Ly6C⁻ TAMs from the primary tumors and metastatic lungs of mice 5 weeks after 4T1 cells inoculation (n=3). The data is normalized to BMDM from healthy mice (n=3).

FIG. 4B shows the immunofluorescent staining of IL-35 (green) and F4/80 (red) in Ly6C⁻F4/80⁺ and Ly6C⁻F4/80⁻ cells from metastatic lungs of 4T1 inoculated mice. Blue, nuclei. Scale bar, 50 μm.

FIG. 4C shows the results of ELISA for measuring IL-35 level in the media collected from the indicated macrophages after 24 hr cultivation. n=3. Data represent mean±S.E.M. *, p<0.05.

FIG. 4D shows the results of RT-qPCR for analyzing the expression of IL2A and EBI3 in CD14⁺ TAMs from metastatic human tumors (n=10) versus peripheral blood monocyte-derived macrophages (PMMs) (n=10). Data represent mean±S.E.M. *, p<0.05.

FIG. 4E shows the results of ELISA for quantification of the secreted IL-35 in the media collected from the indicated macrophages after 24 hr cultivation. n=2. Data represent mean±S.E.M. *p<0.05.

FIG. 4F exhibits the results of transwell migration assay for analyzing migration ability of indicated cancer cell lines upon IL-35 (100 ng/ml) treatment for 48 hr. n=3 Data represent mean±S.E.M. ***, P<0.001.

FIG. 4G shows the results of orthotopic xenograft experiment in Experiment 4. The experiment was conducted by inoculating SAS cells treatment to the tongue of mice. SAS cells were pretreated with recombinant IL-35 (50 ng/ml) or vehicle control for 48 hr before inoculation. IVIS images were taken for visualizing lymph-node 14 days after tumor inoculation (n=6 for each group).

FIG. 4H demonstrates the effect of IL-35 neutralization on metastasis. Upper: schema of the antibody administration in 4T1 orthotopic tumor mouse model (body weight: 15 to 20 g). 2 weeks after tumor implantation, 50 μg IL-35 neutralizing antibody (V1.4C4.22; in 100 μL PBS) or IgG2b isotype control were delivered intratracheally, and total 5 doses of antibodies were given every 3 days. Mice were sacrificed at the end of 4th week. The primary tumors and lungs were harvested for analysis. Middle: photos for primary tumor and representative photos of lungs of mice. Scale bar, 1 cm. Lower: quantification of tumor weight and lung nodules (n=6). Data represent mean±S.E.M. ***, p<0.001.

FIG. 4I illustrates the schema of the antibody therapy experiment. The mice were orthotopically implanted with 4T1 cells, and surgical removal of implanted tumors was performed at the end of 3rd week. The mice (body weight: 15 to 20 g) were injected intraperitoneally (i.p.) with 100 μg anti-IL-35 antibody (V1.4C4.22; in 200 μL PBS), IgG2b isotype control, anti-CSF1R antibody (in 200 μL PBS), and IgG2a isotype control after surgery, then with 50 μg antibodies every 3 days thereafter and a total of 4 dosages were given. IVIS examination was performed at the end of 5th week. n=7 for each group.

FIG. 4J shows the bioluminescence signal of mice treated with the indicated antibodies 2 weeks after surgery (as indicated in FIG. 4I).

FIG. 4K illustrates the percentage of overall survival of tumor-removal mice after antibody administration. The p value is shown in the panel.

FIG. 5A shows the results of the Western blot of IL-12Rβ2, E-cadherin, and N-cadherin in A549 cells treated with TNFα (20 ng/ml) or M1 CM, or control media for 24 hr.

FIG. 5B shows the results of RT-qPCR for analyzing the expression of IL12RB2 in different cancer cell lines upon TNFα (20 ng/ml) treatment for 24 hr. n=2. Data represent mean±S.E.M. *p<0.05; **p<0.01; ***p<0.001.

FIG. 5C illustrates the quantification of the metastatic lung nodules in mice receiving co-injection of 1×10⁶ TNFα-pretreated A549 cells with 5×10⁵ resting (M0) or M2 macrophages. The mice were sacrificed 2 months after tail vein injection (n=6 for each group). Data represent mean±S.E.M. *, p<0.05; **, p<0.01.

FIG. 5D illustrates the results of RT-qPCR (upper) and Western blot (lower) for confirming the knockdown efficiency of IL-12Rβ2 in A549 cells receiving the shRNA against IL12RB2 or a control sequence (pLKO). The number indicates two independent sequences for shRNA experiments. For RT-qPCR, n=2. Data represent mean±S.E.M. *p<0.05; **p<0.01.

FIG. 5E shows the results of the orthotopic tumor experiment in the Experiment 5. 4T1 cells infected with a shRNA against Il12rb2 (shIl12-rβ2) or a control sequence (pLKO) were inoculated to the mice. The tumors and lungs were harvested 4 weeks after tumor implantation. Upper: photos of primary tumors and lungs of tumor-bearing mice. Scale bar, 1 cm. Lower: quantification of tumor weight and lung nodules (n=6 for each group). Data represent mean±S.E.M. **p<0.01.

FIG. 5F illustrates the results of RT-qPCR (upper) and Western blot (lower) for confirming the knockdown efficiency of the IL-12Rβ2 in 4T1 cells receiving the shRNA against Il12rb2 (#1, #2) or a control sequence (pLKO). The number indicates two independent sequences for shRNA experiments. For RT-qPCR, n=3. Data represent mean±S.E.M. *p<0.05; ***p<0.001.

FIG. 5G shows the results of the in vivo metastatic colonization experiment. The GFP-labeled 4T1 cells with Il12rb2 knockdown were co-injected with Ly6C⁻F4/80⁺mTAMs, and the lungs were harvested 5 days after injection (n=5 for each group). Upper: Immunohistochemical (IHC) staining of GFP in representative sections of lungs. Lower: quantification of IHC results by counting the average GFP⁺ colonies from 5 paraffin-embedded lung sections.

FIG. 5H shows the results of Kaplan-Meier survival analysis for showing the prognostic impact of IL12RB2 expression in gastric cancer and lung cancer. The p-value was estimated by log-rank test. The data were obtained from Kaplan-Meier Plotter (http://kmplot.com/).

FIG. 5I shows the results of IHC staining of IL-12Rβ2 in head and neck cancer samples (n=91). Upper: representative IHC images. Scale bar, 100 μm. Lower: a cross-table to show the correlation of IL-12Rβ2 expression and the development of subsequent metastasis in patients (P: primary tumor, M: metastatic tumor). Low H score, 0˜127; high H score, 128˜300.

### DETAILED DESCRIPTION

The present invention is related to pharmaceutical application of IL-35 in cancer metastasis. By using an IL-35 antagonist, the cancer metastasis can be inhibited, reduced, and/or prevented.

As used herein, a “cancer” or “primary cancer” in a subject or patient refers to the presence of cells possessing characteristics typical of cancer-causing cells, such as uncontrolled proliferation, immortality, metastatic potential, rapid growth and proliferation rate, and certain characteristic morphological features. In some circumstances, cancer cells will be in the form of a tumor, or such cells may exist locally within an animal, or circulate in the blood stream as independent cells.

The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

Metastasis may be understood to include micrometastasis, which is the presence of an undetectable amount of cancerous cells in an organ or body part, which is not directly connected to the organ of the original cancerous tumor. Metastasis can also be defined as several steps of a process, such as the departure of cancer cells from an original tumor site, or primary tumor, and migration and/or invasion of cancer cells to other parts of the body. In some aspects, metastasis refers to the subsequent growth or appearance of a cancerous tumor in a different location to an original tumor after treatment of the original tumor.

The term “interleukin-35 antagonist” or “IL-35 antagonist” is referred to as a compound or a substance that acts against or blocks the physiological function of IL-35. Alternatively said IL-35 antagonist could be an antibody-type IL-35 antagonist, a RNAi-type IL-35 antagonist or a small molecule inhibitor. In an alternative embodiment, said antibody-type IL-35 antagonist is an antibody or antigen-binding fragment thereof including but not limited to an antibody or antigen-binding fragment that binds to IL-35, EBI3, subunit P35 of IL-35, EB13/P35 heterodimer, IL-35 receptor on cancer cells, gp130, IL-12Rβ2, IL-27Rα, gp130/IL-12Rβ2 heterodimer, IL-27Rα/IL-12Rβ2 heterodimer, or a combination thereof. In another alternative embodiment, said RNAi-type IL-35 antagonist is a shRNA, a siRNA, a miRNA, Said shRNA, said siRNA, and/or said miRNA is against the expression of IL-35, EBI3, P35, IL-35 receptor, gp130, IL-27Rα, or IL-12Rβ2. In an alternative embodiment, said “small molecule inhibitor” used herein is referred to a compound having inhibitory effect on the physiological function of IL-35.

In an alternative embodiment, said IL-35 antagonist could be a multispecific antibody or antigen-binding fragment. In another embodiment, said IL-35 antagonist is a bispecific antibody or antigen-binding fragment that binds to two antigens selected from a group consisting of IL-35, Epstein-Barr-virus-induced gene3 (EBI3), subunit P35 of IL-35, EBI3/P35 heterodimer, IL-35 receptor on cancer cells, gp130, IL-12Rβ2, gp130/IL-12Rβ2 heterodimer, and CSF1R. In a specific embodiment, said IL-35 antagonist is a bispecific antibody or antigen-binding fragment that binds to IL-35 and CSF1R.

Likewise, the term “Colony stimulating factor 1 receptor antagonist” or “CSF1R antagonist” is referred to as a compound or a substance that acts against or blocks the physiological function of CSF1R. In an alternative embodiment, said CSF1R antagonist is an antibody or antigen-binding fragment thereof binds to CSF1R, or a shRNA, a siRNA, or a miRNA against the expression of CSF1R. In another alternative embodiment, said CSF1R antagonist is a small molecule CSF1R inhibitor.

The term “antibody” encompasses the various forms of antibodies including but not being limited to whole antibodies, antibody fragments, human antibodies, humanized antibodies, chimeric antibodies, T cell epitope depleted antibodies, and further genetically engineered antibodies as long as the characteristic properties according to the invention are retained. “Antibody fragment” comprises a portion of a full-length antibody, preferably the variable domain thereof, or at least the antigen binding site thereof.

Examples of antibody fragments include diabodies, single-chain antibody molecules, and multispecific antibodies formed from antibody fragments. scFv antibodies are, e.g., described in Houston, J. S., Methods in Enzymol. 203 (1991) 46-88). In addition, antibody fragments comprise single chain polypeptides having the characteristics of a VH domain binding to an antigen, namely being able to assemble together with a VL domain, or of a VL domain binding to an antigen, namely being able to assemble together with a VH domain to a functional antigen binding site and thereby providing the property.

The antibody of the present invention may be modified by attachment with various molecules such as an enzyme, a fluorescent material, a radioactive material and a protein. The modified antibody may be obtained by chemically modifying the antibody. This modification method is conventionally used in the art. Also, the antibody may be obtained as a chimeric antibody having a variable region derived from a non-human antibody, and a constant region derived from a human antibody, or may be obtained as a humanized antibody including a complementarity-determining region derived from a non-human antibody, and a framework region (FR) and a constant region derived from a human antibody. Such an antibody may be prepared by using a method known in the art.

The description of “against the expression” used herein means to reducing, stopping, preventing the transcription or translation of a target protein or gene. Commonly used tool for against the expression of a target protein or gene includes but not limited to shRNA, a siRNA, or miRNA.

“Primary cancer treatment”, as used herein, means any treatment of any kind or means intended to or having the effect of partially or completely removing, destroying, damaging, excising, reducing in size, rendering benign or inhibiting the growth of, a cancer or tumor. For example, primary treatment may include one or more of endocrine therapy, chemotherapy, radiotherapy, hormone therapy, surgery, gene therapy, thermal therapy, and ultrasound therapy. In an alternative embodiment the primary cancer treatment is surgical excision of a solid tumor.

The terms “administer”, “administering”, or “administration” as used herein is referred to implanting, absorbing, ingesting, injecting, inhaling, or otherwise introducing an inventive pharmaceutical composition described herein.

The description “treating cancer metastasis” used herein is referred to inhibiting, reducing, and/or preventing cancer metastasis. Specifically, in an alternative embodiment, said inhibiting cancer metastasis means inhibiting the progression or development of cancer metastasis. In another embodiment, said reducing cancer metastasis, means reducing the degree, area, or amount of cancer metastasis. In another embodiment, said preventing cancer metastasis means preventing the occurrence or recurrence of cancer metastasis.

“An effective amount” or “a therapeutically effective amount” used herein is referred to the amount of each active agent required to confer the desired effect (ex. treating cancer metastasis is the desired effect of the present invention) on the subject, either alone or in combination with one or more other active agents. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In the first aspect of the present invention, a method for treating cancer metastasis is provided. The present method for treating cancer metastasis comprises administering a subject in need a therapeutically effective amount of an IL-35 antagonist.

Said therapeutically effective amount is defined as set forth in the preceding paragraphs. In an alternative embodiment of the present invention, said therapeutically effective amount can be determined from an animal model experiment or from a human clinical trial; for instance, as taught by Guidance for Industry (FDA, 2005, page 7, Table 1). In some embodiment, said therapeutically effective amount of said IL-35 antagonist is 0.01 to 20 mg/kg body weight of the subject. In a preferable embodiment, said therapeutically effective amount might be any range between the following numerals: 0.01, 0.05, 0.1, 0.3, 0.5, 1, 1.5, 2, 3, 4, 5, 10, 12, 14, 16, 18, 20 mg/kg body weight of the subject.

In a preferable embodiment, the present method further comprises administering said subject a therapeutically effective amount of a CSF1R antagonist. In an alternative embodiment, said therapeutically effective amount of said CSF1R antagonist is 0.01 to 20 mg/kg body weight of the subject. In a preferable embodiment, said therapeutically effective amount might be of a range between any two of the following numerals: 0.01, 0.05, 0.1, 0.3, 0.5, 1, 1.5, 2, 3, 4, 5, 10, 12, 14, 16, 18, 20 mg/kg body weight of the subject.

In some embodiment, said IL-35 antagonist and said CSF1R antagonist could be administered simultaneously or at any interval with each other. In an alternative embodiment, said interval might be 1, 3, 5, 10, 30, 60, 120, 240, or 600 minutes. In an alternative embodiment, said IL-35 antagonist is administered first and said CSF1R antagonist is administered thereafter. In another embodiment, said CSF1R antagonist is administered first and said IL-35 antagonist is administered thereafter.

In some embodiments, dosing frequency of said IL-35 antagonist and/or said CSF1R antagonist is twice every week, once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, conventional methods, known to those of ordinary skill in the art of medicine, can be used to administer said IL-35 antagonist and/or said CSF1R antagonist to the subject, depending upon the type of cancer to be treated. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods.

In the second aspect of the present invention, a use of IL-35 antagonist in preparing a pharmaceutical composition for treating cancer metastasis is provided. In the third aspect of the present invention, said pharmaceutical composition is provided.

Said pharmaceutical composition comprises IL-35 antagonist and a pharmaceutically acceptable carrier. The term “pharmaceutically acceptable” used herein is referred to the meaning known in the field. For instance, said “pharmaceutically acceptable” means non-toxic to the subject and having no interference with the efficacy of the active ingredient of the pharmaceutical composition at issue. Said pharmaceutically acceptable carrier includes but not limit to water, PBS, salt solutions, gelatins, oils, alcohols, or a combination thereof. Said pharmaceutical composition may further comprise a pharmaceutically acceptable excipient. Said excipient includes but not limits to a disintegrating agent, a binder, a lubricant, a preservative, or a combination thereof.

Said IL-35 antagonist could contain a suitable percentage of said pharmaceutical composition. It is known in the art that the amount of the active ingredient in a pharmaceutical composition can be determined based on several factors, such as: stability of the active ingredient, efficacy of the active ingredient (corresponding to the effective amount of the active ingredient), regimen of the medical practitioner, and patient compliance. In an alternative embodiment, said pharmaceutical composition comprises 0.1 to 100 mg/mL of said IL-35 antagonist based on the total weight of said pharmaceutical composition. In another alternative embodiment, the amount of said IL-35 antagonist is a range between any two of the following numerals: 0.1, 0.2, 0.3, 0.4, 0.5, 1, 2, 3, 4, 5, 10, 15, 20, 25, 30, 35, 40, 45, 50, 55, 60, 65, 70, 75, 80, 85, 90, 95, 100 mg/mL.

In a preferable embodiment, said pharmaceutical composition further comprises a CSF1R antagonist. Preferably, said pharmaceutical composition comprises 0.1 to 100 mg/mL of said CSF1R antagonist based on the total weight of said pharmaceutical composition. In another alternative embodiment, the amount of said CSF1R antagonist is a range between any two of the following numerals: 0.1, 0.2, 0.3, 0.4, 0.5, 1, 2, 3, 4, 5, 10, 15, 20, 25, 30, 35, 40, 45, 50, 55, 60, 65, 70, 75, 80, 85, 90, 95, 100 mg/mL.

In the fourth aspect of the present invention, a kit for treating cancer metastasis of a subject had a primary cancer treatment is provided. Said kit comprises a first container comprising an IL-35 antagonist; and a second container comprising a CSF1R antagonist.

Said “a subject had a primary cancer treatment” means said subject had a primary cancer but was treated by a cancer treatment. Said primary cancer treatment is defined as set forth in the preceding paragraphs.

In a preferable embodiment, said first container comprises the pharmaceutical composition of the present invention as described above. In an alternative embodiment, said IL-35 antagonist and/or said a CSF1R antagonist contained in the aforesaid container are formulated according to the requirements of storage stability, administration route, etc. For instance, the IL-35 antagonist contained in said first container could be formulated as an injection and said first container is an ampoule. In the embodiment that said IL-35 antagonist and/or said a CSF1R antagonist are formulated as injections, said kit might further comprise a syringe.

In the fifth aspect of the present invention, a method for treating cancer, comprising (a) administering a subject in need with a primary cancer treatment; and (b) administering said subject a therapeutically effective amount of an IL-35 antagonist and/or a therapeutically effective amount of a CSF1R antagonist.

In an alternative embodiment, said step (a) and said step (b) are conducted at any interval. For instance, 30 minutes, 60 minutes, 5 hours, 10 hours, 20 hours, 1 days, 3 days, 5 days, 1 week, 3 weeks, 1 month, 3 months, or 6 months.

In a preferable embodiment, said subject is administered with both of said IL-35 antagonist and said CSF1R antagonist in said step (B); wherein said IL-35 antagonist and said CSF1R antagonist are administered simultaneously or at any interval with each other. Said interval is defined as set forth in the preceding paragraphs.

In order that the invention described herein may be more fully understood, the following examples/experiments are set forth. It should be understood that these examples are for illustrative purposes only and are not to be construed as limiting this invention in any manner.

### Experiment 1: Characterization of Tumor-Associated Macrophages in Primary and Metastatic Tumors

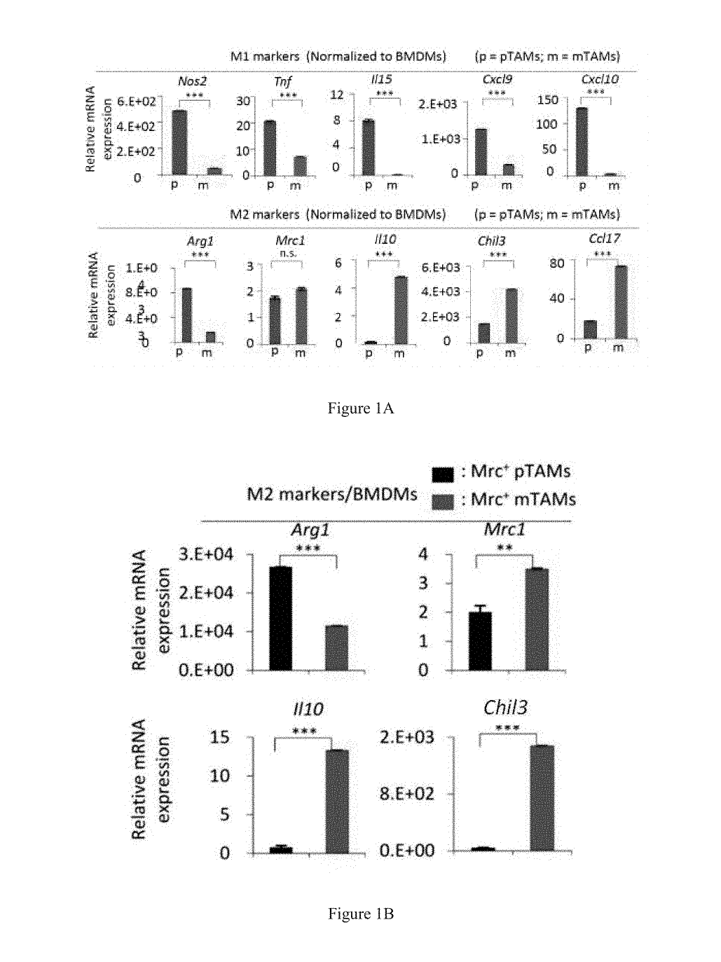

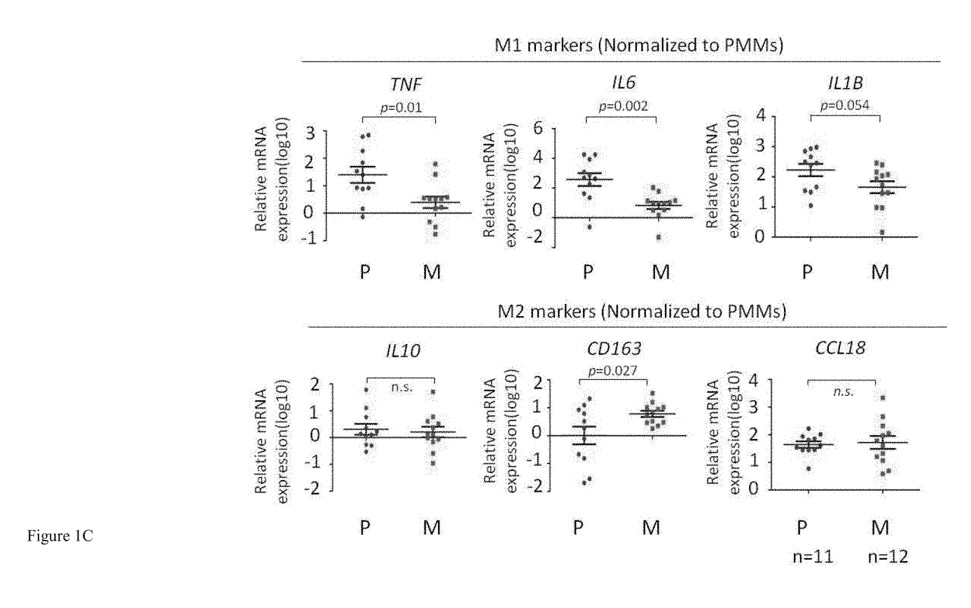

In this study, the distinct roles of tumor associated macrophages in primary and metastatic tumor (i.e., pTAM and mTAM) were investigated. We generated a murine orthotopic breast cancer model by inoculating syngeneic 4T1 mammary cancer cells into BALB/c mice. Pulmonary metastases developed four to five weeks after the inoculation of tumor cells. CD11b⁺F4/80⁺ macrophages were isolated from the primary tumors and metastatic lung tissues for further analyses. The results showed that the pTAMs primarily expressed M1 macrophage-associated markers. Interestingly, they also expressed certain M2 macrophage-associated markers (e.g., Arg1 and Mrc1). In contrast, a predominant M2 pattern but not M1 was noted in the mTAMs (FIG. 1A). In addition, we isolated the F4/80⁺Mrc1⁺ TAMs from primary and metastatic tumors. In this population, the mTAMs still expressed higher levels of M2-associated markers than the pTAMs (FIG. 1B). Immunohistochemical (IHC) staining for M1 and M2 markers in the harvested primary and metastatic tumors confirmed the findings (data not shown).

Next, we isolated CD14⁺ TAMs from human primary and metastatic cancers and examined the expression of immunologic markers. A consistent result was noted: the pTAMs expressed M1-specific markers (e.g., TNF, IL6, and IL1B), and the mTAMs were more likely to express higher levels of M2 markers than the pTAMs (e.g., CD163) (FIG. 1C). Collectively, these results suggest that the pTAMs and mTAMs are separate populations that express distinct markers and harbor differential functions.

### Experiment 2: mTAMs Facilitate the Colonization of Metastatic Tumors

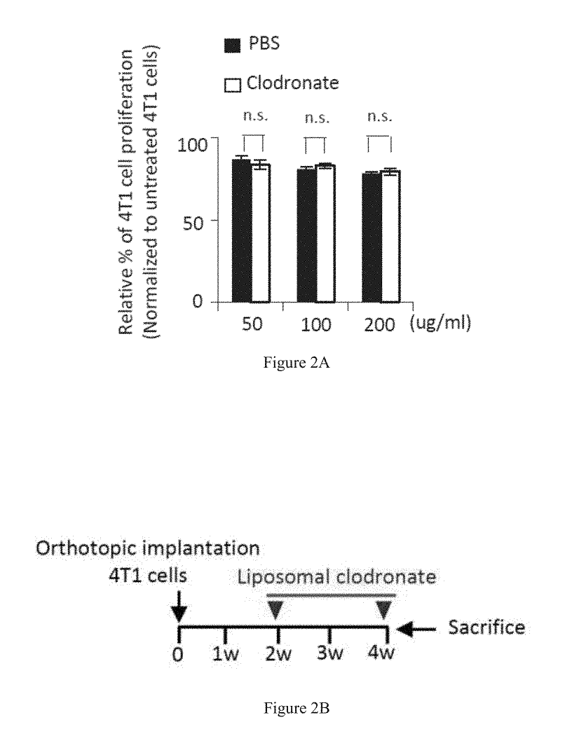

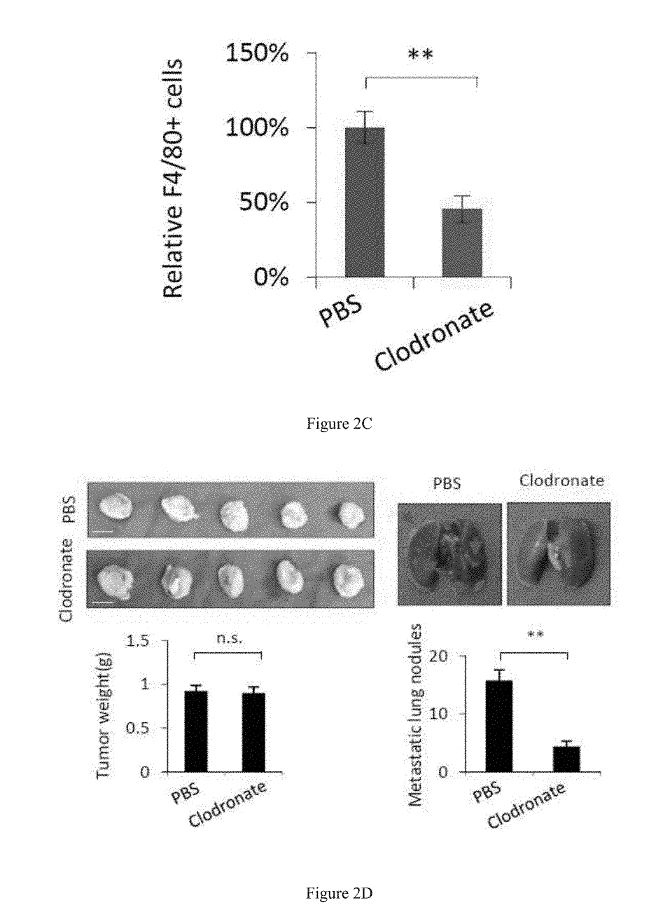

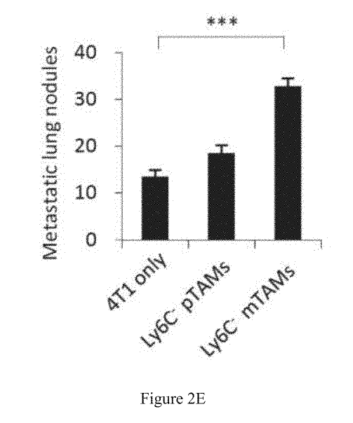

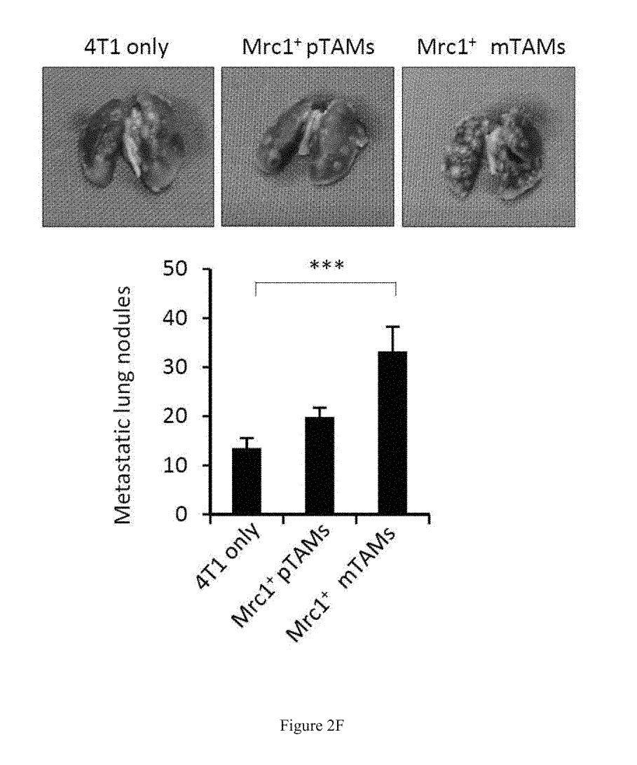

In this study, we speculated that mTAMs participate in metastatic colonization and thus focused on the role of macrophages at the metastatic sites. It is known in the file that the depletion of macrophages can be achieved by liposomal clodronate (Qian et al., 2009; Pallasch et al., 2014). Thus, we depleted the pulmonary macrophages by intratracheal injection of liposomal clodronate and observed the impact on metastatic colonization. Clodronate itself did not have a significant impact on the proliferation of 4T1 cells (FIG. 2A). Ablation of pulmonary macrophages significantly reduced pulmonary metastasis without affecting primary tumor weights (FIG. 2B-D).

We next investigated the pro-colonization effect of pTAMs and mTAMs. Co-injection of 4T1 cells with CD11b⁺F4/80⁺Ly6c⁻ mTAMs via tail vein increased the colonization of lung tumors compared to the co-injection with CD11b⁺F4/80⁺Ly6c⁻ pTAMs (FIG. 2E). Consistent results were demonstrated by the co-injection of 4T1 cells with F4/80⁺Mrc1⁺ pTAMs or mTAMs via tail vein. The F4/80Mrc1⁺mTAMs also had a greater ability to promote pulmonary colonization than pTAMs (FIG. 2F). Altogether, these results suggest that mTAMs harbor a greater ability to facilitate metastatic colonization.

### Experiment 3: M2-Like Macrophages Promote Epithelial Phenotypes of Cancer Cells

Next, we investigated whether the mTAMs are able to directly influence the colonization of cancer cells. Accumulated evidence supports the role of mesenchymal-epithelial transition (MET) in metastatic colonization (Tsai et al., 2012). We investigated whether the mTAMs regulate epithelial plasticity of cancer cells and performed an in vitro experiment to rule out the effect of other immune cells.

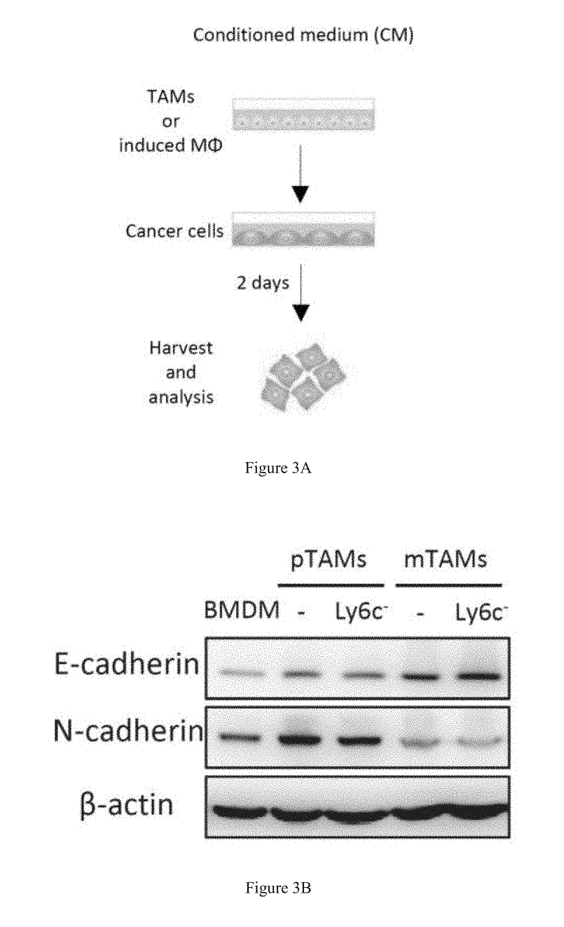

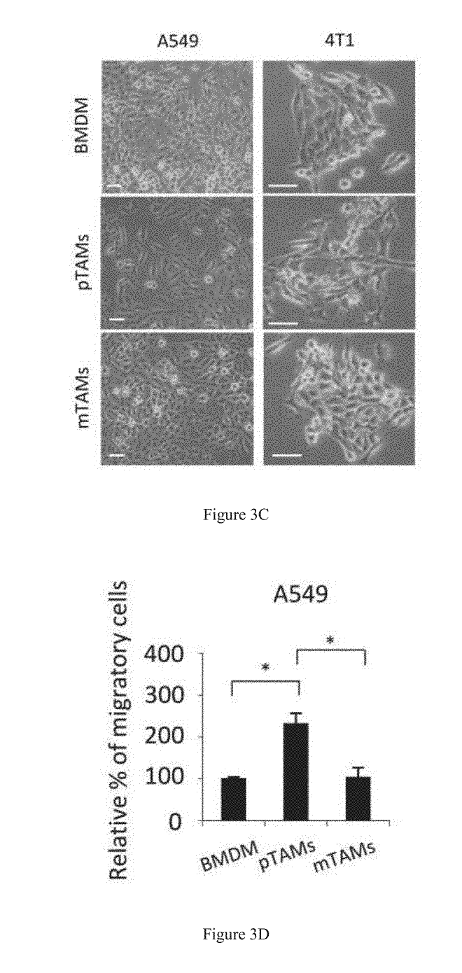

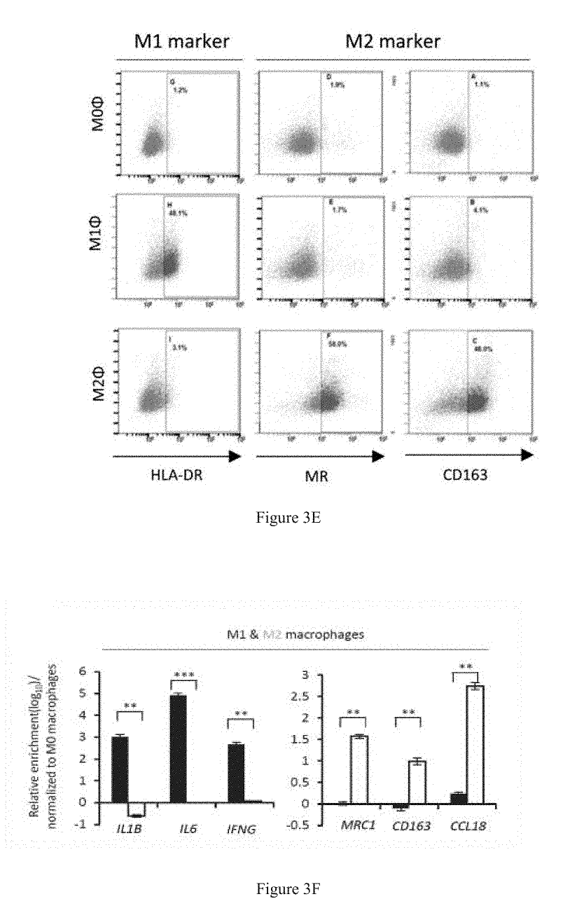

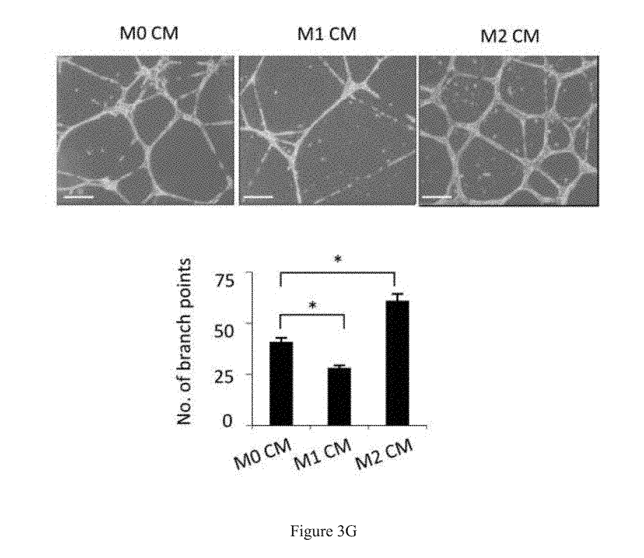

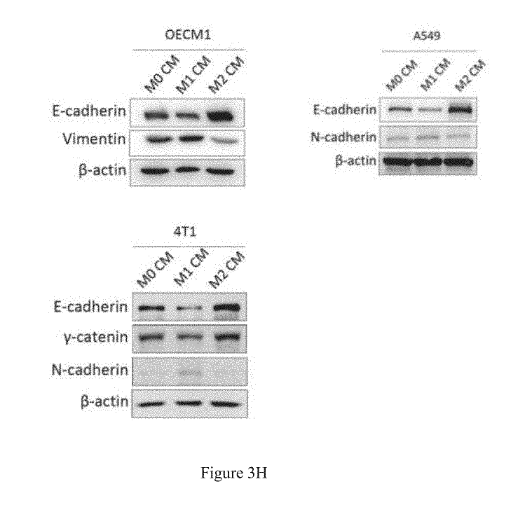

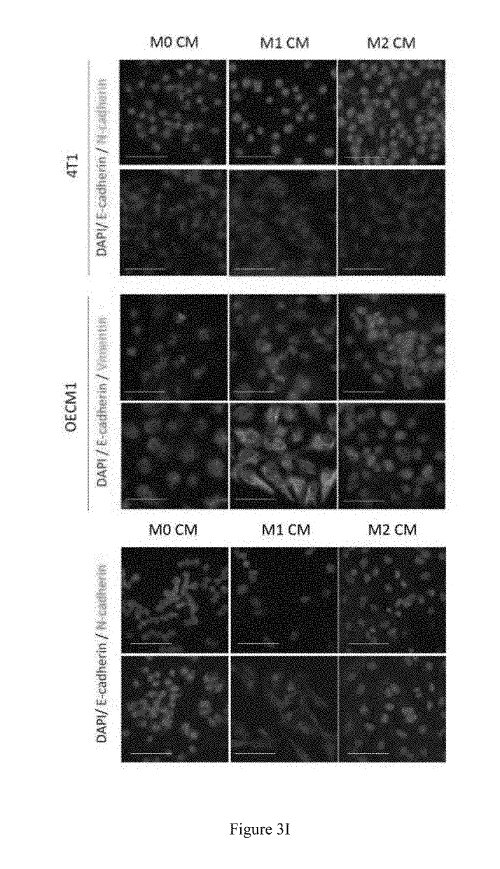

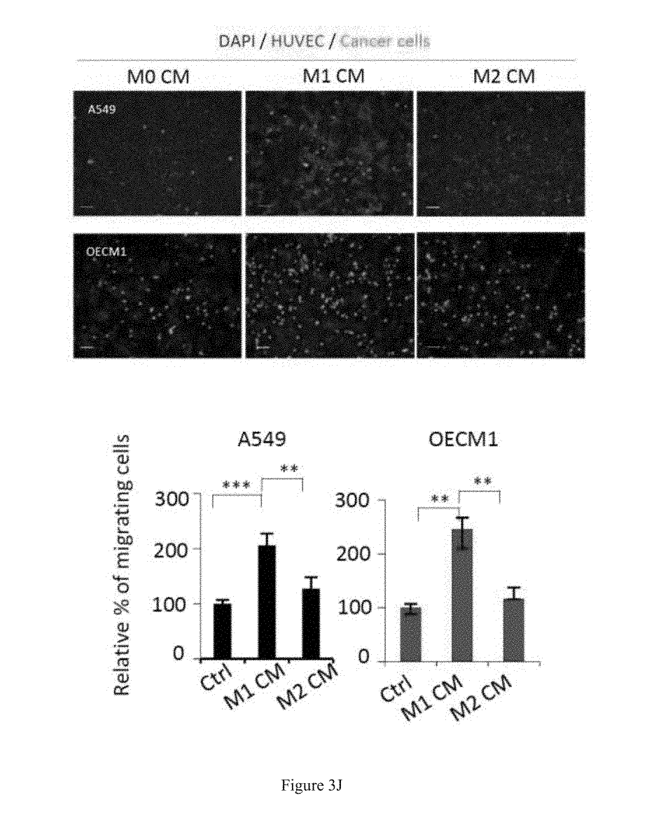

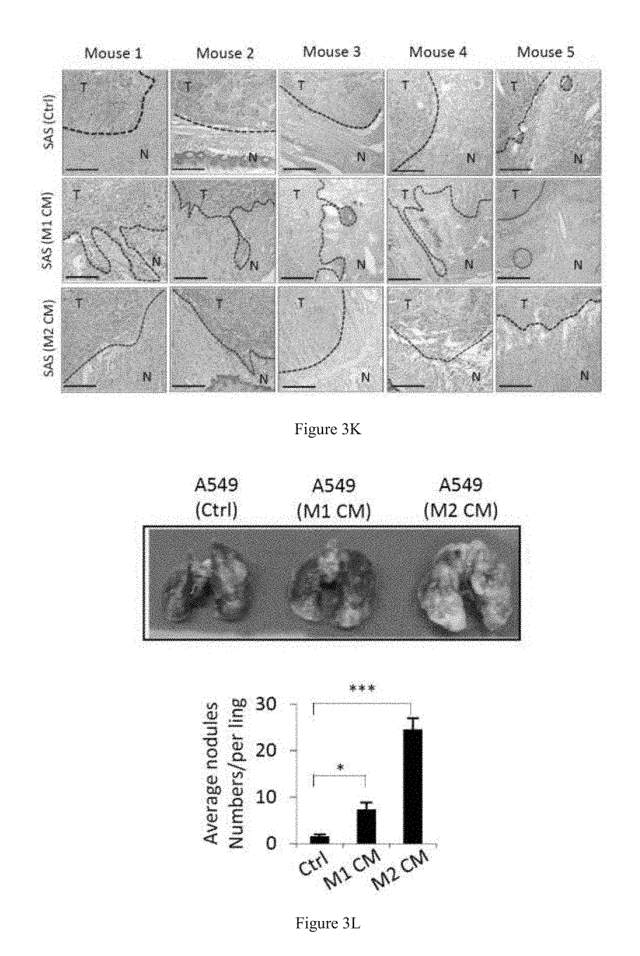

To this end, we isolated pTAMs and mTAMs from the 4T1 orthotopic model and then treated cancer cells (A549 cells and 4T1 cells) with conditioned media from TAM (FIG. 3A). Compared with the effect of the pTAMs, treatment of 4T1 cells with medium from the mTAMs increased the expression of E-cadherin and downregulated N-cadherin (FIG. 3B). The medium from mTAMs also induced an epithelial morphology and reduced the migration of cancer cells (FIG. 3C and FIG. 3D), suggesting that the mesenchymal phenotype was suppressed upon treatment with conditioned medium from the mTAMs.

Since the mTAMs predominantly showed a M2-like phenotype (previously shown), we performed in vitro polarization of human CD14⁺ monocytes into M1-like and M2-like macrophages for subsequent experiments. A standard polarization procedure was conducted according to previous reports (Martinez et al., 2006; Kzhyshkowska et al., 2008; Park et al., 2009). Analyses of surface markers, gene expression profiles, and angiogenic capability confirmed the successful polarization of macrophages (FIG. 3E, FIG. 3F, FIG. 3G).

We further performed cDNA microarray analysis to generate a transcriptome profile of lung cancer cell line A549 treated with conditioned media from M1 or M2 macrophages (M1-CM, M2-CM; data not shown). A Gene Set Enrichment Analysis (GSEA) showed that the gene expression signature of the M1-CM-treated cancer cells was significantly correlated with the core epithelial-mesenchymal transition (EMT) signature. In contrast, an inverse correlation between the M2-CM-treated signature and EMT signature was found (data not shown), suggesting that the M2 secretome influences the cancer cells to acquire an epithelial phenotype and undergo reverse EMT.

Consistently, compared with the conditioned medium from the M1 macrophages, the medium from the M2 macrophages induced MET in different cancer cell lines, which was demonstrated by the upregulation of epithelial markers and downregulation of mesenchymal markers (FIG. 3H), an epithelial morphology with membranous expression of E-cadherin (FIG. 3I) and a reduced ability for penetration through the endothelium (FIG. 3J).

Because the inhibition of EMT reduces local invasion but facilitates metastatic colonization (Yan et al., 2010; Tsai et al., 2012), we performed two experiments to validate the effect of the M2-CM-regulated epithelial plasticity of cancer cells in vivo. First, we treated oral cancer cell line SAS with M1, M2, or control media and then inoculated SAS cells on the tongues of mice. The results demonstrated an increase in local invasion with the M1-CM-treated SAS cells, however, the M2-CM-treated cells formed localized tumor without peripheral invasion (FIG. 3K).

Next, we injected M1- or M2-CM-treated A549 cells via tail vein to investigate the ability of metastatic colonization. A significant increase in the numbers of metastatic nodules was noted in the group of mice that received the M2-CM-treated cancer cells (FIG. 3L). Together, these results suggest that M2-like macrophages suppress EMT and promote metastatic colonization of cancer cells.

### Experiment 4: Metastatic TAMs Secrete IL-35 to Promote the Colonization of Cancer Cells

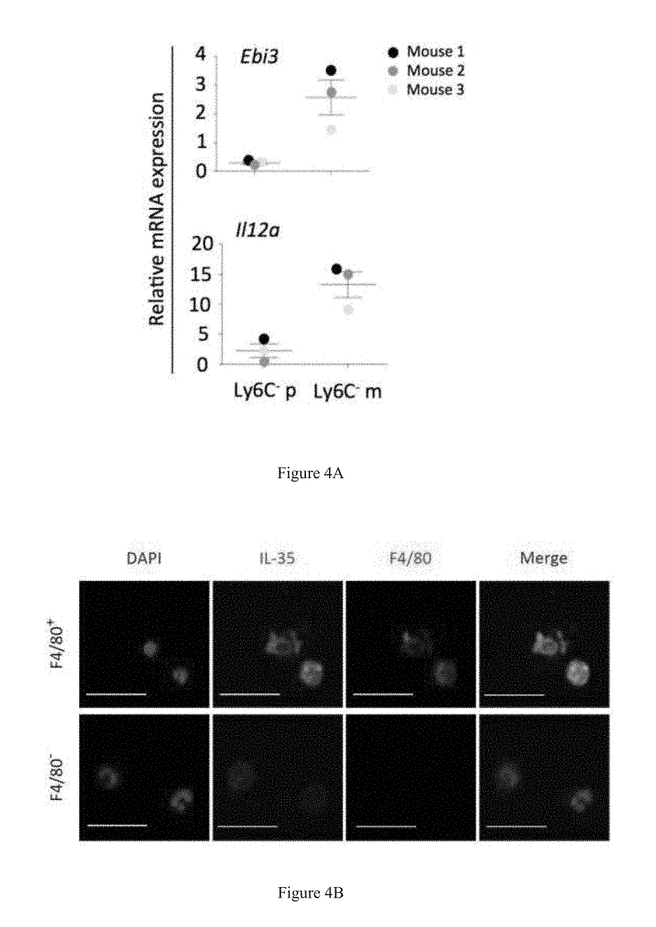

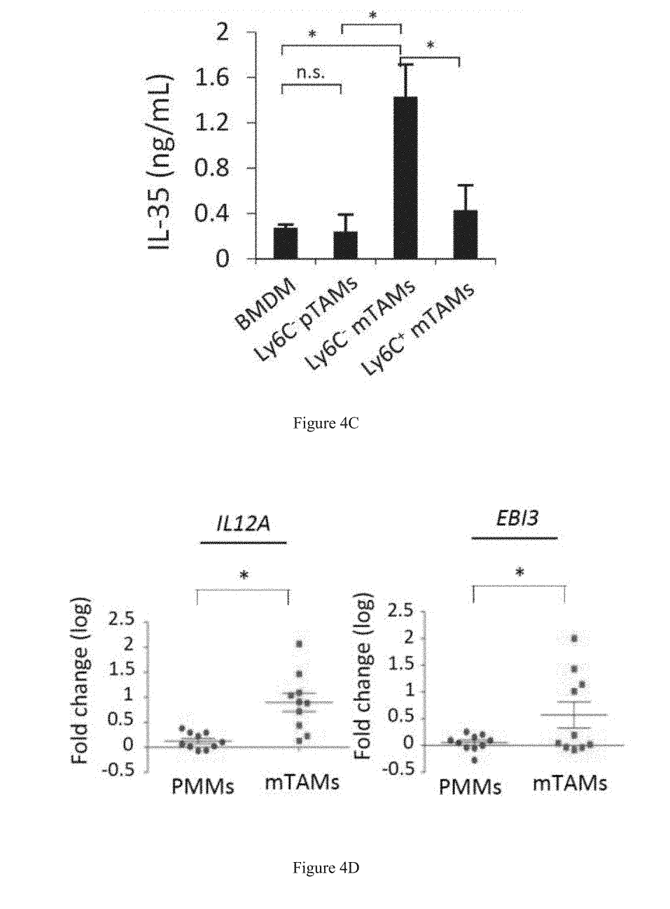

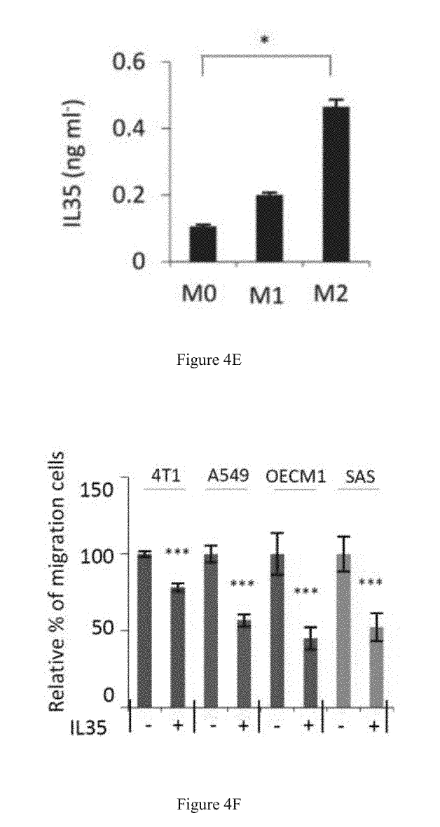

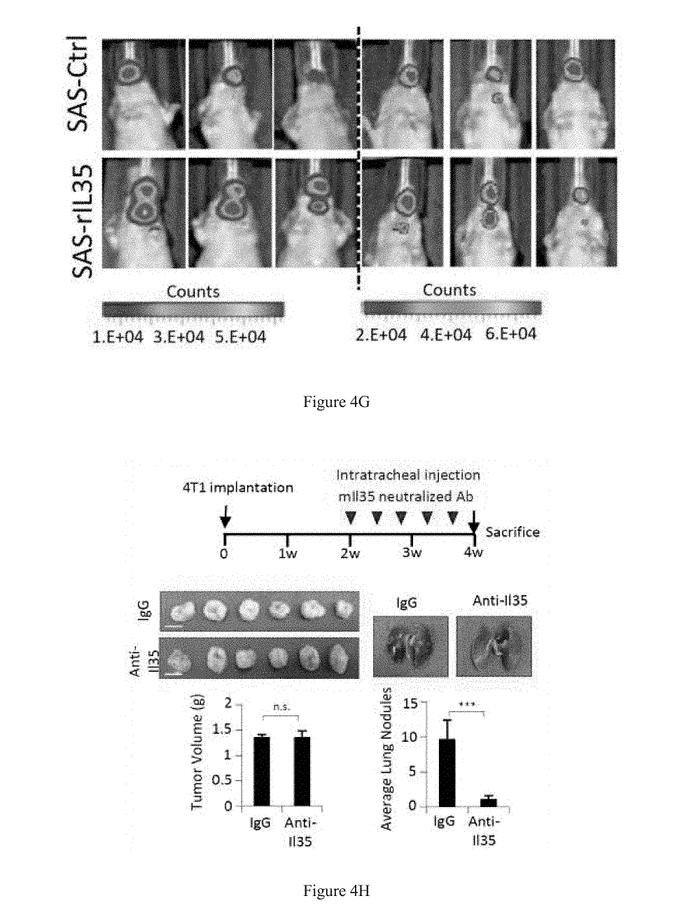

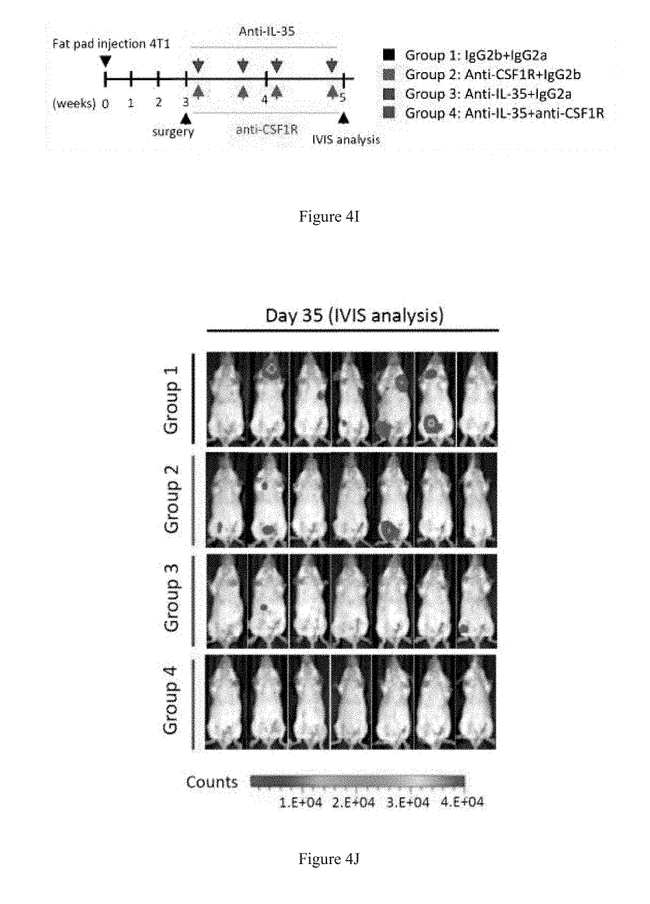

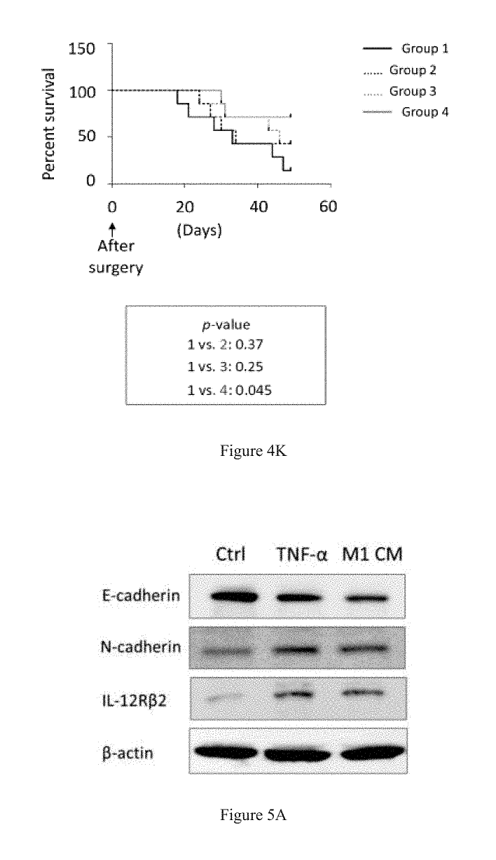

To elucidate the factors involved in mTAM-mediated cancer colonization, we performed microarray analysis of pTAM and mTAM isolated from 4T1 orthotopic tumor model. IL-35 (composed of Ebi3 and IL-12A) was found as the secreted factor upregulated in mTAM. In the 4T1 mouse tumor model, a significantly elevated expression of Ebi3 and Il12a was noted in the mTAMs but not pTAMs compared with the bone marrow-derived macrophages (BMDMs) (FIG. 4A). In the lungs of these mice, expression of IL-35 was noted in the F4/80⁺ TAMs but not in the F4/80⁻ cells, confirming the source of IL-35 expression in the metastatic tumors (FIG. 4B). The Ly6C⁻mTAMs secrete higher levels of IL-35 compared with the pTAMs and BMDMs (FIG. 4C).

In the human cancer samples, the CD14⁺ TAMs from metastatic tumors expressed higher levels of EBI3 and IL12A compared with peripheral blood monocyte-derived macrophages (PMMs) (FIG. 4D). The in vitro polarized human M2 macrophages expressed and secreted high levels of IL-35 (FIG. 4E). Furthermore, it was noted that IL-35 pretreatment decreased migration ability of 4T1, A549, OECM1, and SAS cells (FIG. 4F). Inoculation of IL-35-pretreated SAS cells on the tongues of nude mice increased metastatic colonization of tumor cells (FIG. 4G). Consistently, intratracheal injection of IL-35 neutralizing antibody in 4T1-tumor bearing mice significantly reduced lung metastasis without affecting the growth of the primary tumors (FIG. 4H). The anti-IL-35 antibody did not have any effect on the proliferation of the 4T1 cells (data not shown).

To validate the role of IL-35 in metastatic colonization, we removed the primary tumor in 4T1 mice model 3 weeks after implantation. After surgery, the mice were administered with antibodies against IL-35, CSF1R, or both, or IgG isotype control (FIG. 4I). The development of metastasis was monitored by IVIS analysis 2 weeks after surgery. Administration of either anti-IL-35 or anti-CSF1R antibody reduced the development of metastasis, and combination of the anti-IL-35 and anti-CSF1R antibodies yielded the best improving effect on preventing metastasis (FIG. 4J). Anti-IL-35 antibody treatment or anti-CSF1R antibody treatment increased the survival rate of the mice. Among them, mice received combination treatment (anti-IL-35 and anti-CSF1R) displayed the best improving survival rate (FIG. 4K). Collectively, these results indicate that macrophage-secreted IL-35 play an important role in metastatic colonization of various kinds of cancer cells. Moreover, neutralization of IL-35 can reduce metastasis and improve survival rate in mice.

### Experiment 5: TNFα Induces IL-12Rβ2 Expression in Cancer Cells to Promote Metastatic Colonization

We next investigated the expression of the IL-35 receptor on cancer cells for receiving the signals from TAMs. The IL-35 receptor is a heterodimer comprising IL-12Rβ2 and gp130 (Collison et al., 2012). Tumor necrosis factor (TNF)-α, an inflammatory cytokine produced by macrophages, can induce EMT (data not shown). We examined whether TNFα-primed cancer cells harbor IL-35 receptor to receive signals at the metastatic sites.

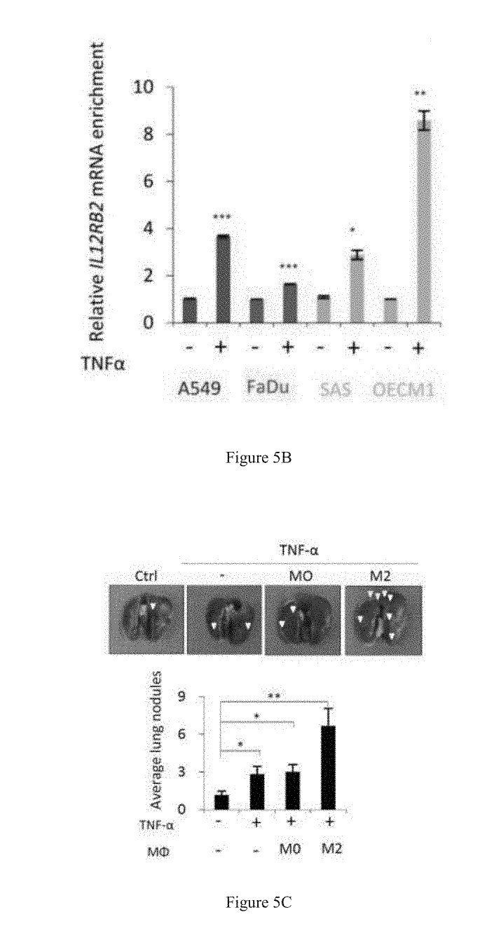

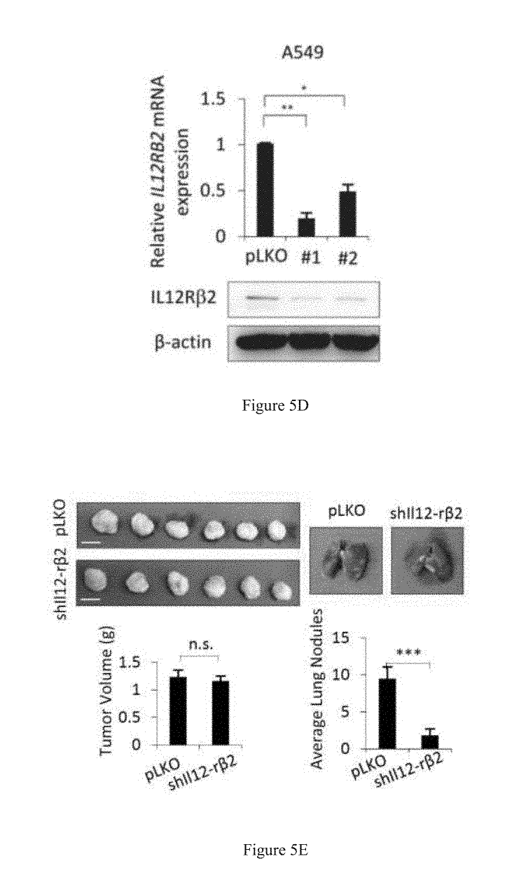

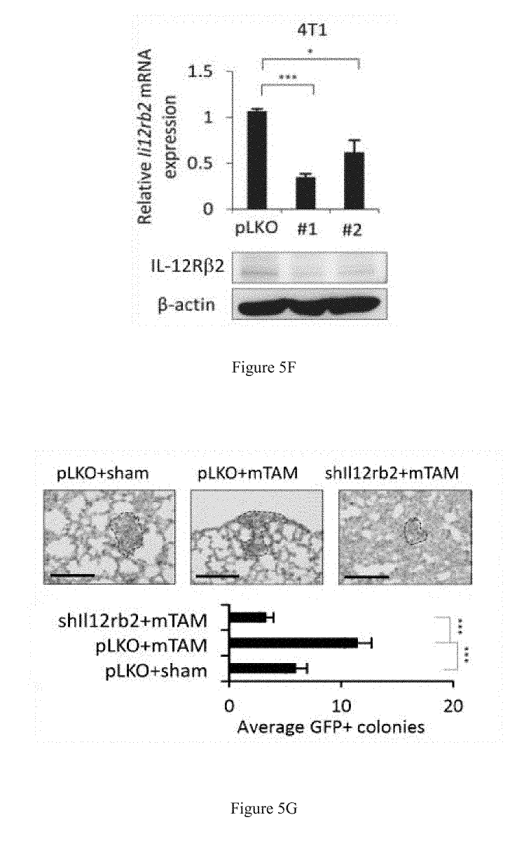

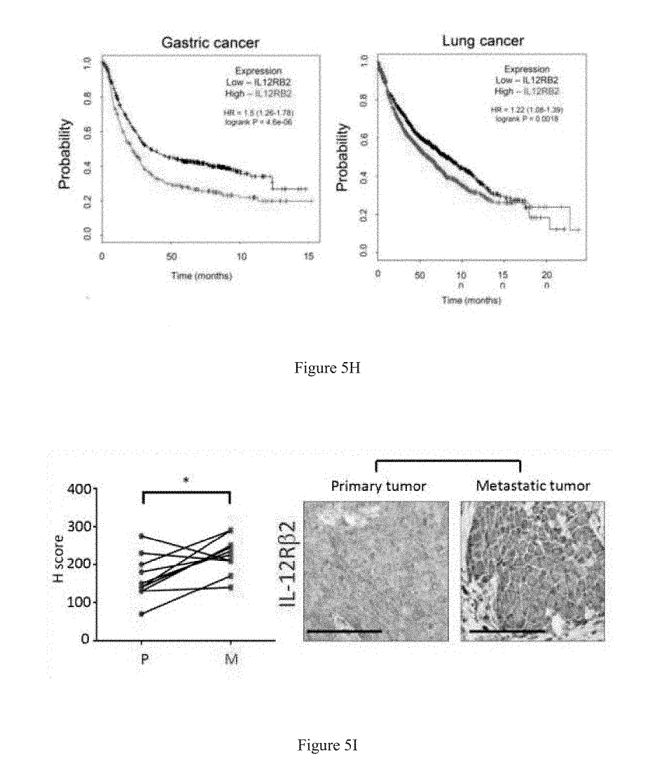

M1-CM or TNFα treatment induced EMT marker (N-cadherin) and IL-12Rβ2 expression in A549 cells (FIG. 5A). In addition, TNFα upregulated the level of IL12Rβ2 mRNA in four different cancer cell lines (FIG. 5B). Next, we elucidated the role of TNFα-primed cancer cells on metastatic colonization through the IL-35-mediated signal. TNFα-pretreated A549 cells had a higher capability for pulmonary colonization. Co-injection with M2 macrophages significantly enhanced the colonization of TNFα-primed cancer cells (FIG. 5C and FIG. 5D). In the 4T1 syngeneic tumor model, the suppression of IL-12Rβ2 expression reduced metastasis without affecting the growth of the primary tumor (FIG. 5E and FIG. 5F). Moreover, mTAM-induced metastatic colonization was abrogated in Il12rb2-knockdown tumor cells (FIG. 5G).

Analyses of public databases revealed that high levels of IL12Rβ2 in cancer samples were associated with worse survival of lung cancer and gastric cancer patients (FIG. 5H). IHC examination of the expression of IL-12Rβ2 in primary tumors of 91 head and neck cancers showed that high levels of IL-12Rβ2 correlated with a higher probability of subsequent development of metastasis (Table 1). Moreover, analysis of the expression of IL-12Rβ2 in 10 matched primary-metastatic tumor sample pairs revealed that the expression of IL-12Rβ2 was higher in the metastatic tumors (FIG. 5I).

TABLE 1

Metastasis

| IL12Rβ2 | No | Yes | Total | P value |
|---|---|---|---|---|
| Low | 29 | 17 | 46 | 0.016 |
| High | 17 | 28 | 45 | |
| Total | 46 | 45 | 91 | |

Collectively, these data indicate that inflammation-induced EMT upregulates the expression of IL-12Rβ2 in cancer cells, which is critical for the cancer cells to respond to IL-35 from mTAMs for completing metastatic colonization.

### Materials and Methods

Cell Lines, Plasmids, and Reagents.

The human head and neck cancer cell line SAS (RID # CVCL_1675), human embryonic kidney cell line 293T (ATCC CRL-11268), human lung cancer cell line A549 (ATCC CCL-185), BALB/c mouse breast carcinoma cell line 4T1 (ATCC CRL2539), and C57BL/6 mouse lung carcinoma cell line LLC1 (ATCC CRL-1642) were originally from ATCC. The human head and neck cancer cell line OECM1 was provided by Dr. Kuo-Wei Chang (National Yang-Ming University of Taiwan). The pLKO.1-control (ASN0000000004), hIL12Rβ2#1 shRNA (TRCN0000436750), hIL12Rβ2#2 shRNA (TRCN0000058158), mIL12Rβ2#1 shRNA (TRCN0000067720), and mIL12Rβ2#2 shRNA (TRCN0000067721) were obtained from the National RNAi Core Facility of Taiwan for gene silencing. Recombinant human interferon-γ (IFN-γ), interleukin-4 (IL-4), macrophage colony-stimulating factor (M-CSF), and Granulocyte-macrophage colony-stimulating factor (GM-CSF) were purchased from PeproTech (Rocky Hill, N.J.). Recombinant human TNFα was purchased from Abbiotec (cat. no. 600173, Abbiotec, Inc., San Diego, Calif.). Recombinant human IL-35 was purchased from BioLegend (cat. no. 578502, BioLegend, Inc., San Diego, Calif.). Lipopolysaccharide (LPS) and dexamethasone, were purchased from Sigma-Aldrich (St. Louis, Mo.).

Animal Model.

The animal experiment was approved by the Institutional Animal Care and Utilization Committee of Taipei Veterans General Hospital (IACUC 2016-115). We used three models to monitor the development of metastasis. For the syngeneic and orthotopic tumor models of mice, 1.5×10⁵ 4T1 cells were inoculated into the fat pad of 5- to 6-week-old BALB/c mice. For the syngeneic model, 1.5×10⁵ LLC cells were inoculated subcutaneously into C57BL/6 mice. For the orthotopic xenotransplantation model, 1×10 SAS cells were implanted into the tongue of 6-week-old nude mice. After 4˜5 weeks, metastatic lung nodules were examined in the 4T1 and LLC models, and metastatic lymph nodes in the xenograft SAS model were examined with a Xenogen IVIS spectrum system.

Metastatic Colonization.

For assaying the ability for metastatic colonization, cancer cells carrying luciferase vectors were suspended and injected into tail vein of mice. Lung metastases were measured by lung surface nodules, GFP-staining on lung paraffin sections, or ex vivo imaging with the IVIS system. Liposomal clodronate was intraperitoneally injected for systemic depletion of macrophages or intratracheally injected for depletion of pulmonary macrophages. Antibodies for intercepting the metastatic signals were also delivered intraperitoneally or intratracheally. For investigating the effect on colonization of the micrometastases, primary breast tumors of the 4T1 orthotopic model were surgically removed 3 weeks after inoculation, and IVIS spectrum imaging was used to confirm the complete removal of tumors. After surgery, the mice were treated with antibodies, inhibitors, or control as indicated in each figure. The recurrent/metastatic tumors were visualized by IVIS imaging, and the survival of mice was estimated through the Kaplan-Meier method.

Isolation of TAMs from Mice and Human Tumors.

TAMs were isolated from fresh primary and metastatic tumor samples. Briefly, the tissues were minced into small pieces and digested with Dulbecco's modified Eagle's medium containing 1.5 mg/ml collagenase IV (no. 9001-12-1, Thermo Fisher Scientific Inc., Waltham, Mass.) and 1.5 mg/ml hyaluronidase (no. H6254, Sigma-Aldrich, St. Louis, Mo.) at 37° C. for 1 hr. The cells were subsequently filtered through a 200 μm cell strainer. The cells were then centrifuged at 700 g for 20 min, and Percoll (no. 17-5445-02, Sigma-Aldrich, St. Louis, Mo.) was used to separate the different layers of cells. Human TAMs were isolated by a magnetic-activated cell sorting (MACS) using CD14 microbeads (no. 130-050-201, Miltenyi Biotec GmbH, Bergisch Gladbach, Germany), and mouse TAMs were sorted with the indicated markers shown in the figures using a BD FACSAria cell sorter (BD Biosciences, San Jose, Calif.).

Patient Samples.

The study was approved by the Institutional Review Board (2016-07-001CC) of Taipei Veterans General Hospital. Three sets of patient samples were used in this study. The first set comprised paraffin-embedded samples of 10 primary and 10 metastatic tumors from the same patients with head and neck cancers. These samples were used for IHC analysis for IL-12Rβ2. The second set comprised 11 freshly isolated primary tumors (6 colon cancer, 4 head and neck cancer, and 1 gastric cancer) and 12 freshly isolated metastatic tumors (7 colon cancer, 4 head and neck cancer, and 1 gastric cancer). The samples were digested with Dulbecco's modified Eagle's medium containing 1.5 mg/ml collagenase IV and 1.5 mg/ml hyaluronidase immediately after harvesting from surgery, and MACS was used to sort the CD14⁺ TAMs for subsequent analysis. Human peripheral blood monocyte-derived macrophages (PMMs) were polarized from peripheral blood mononuclear cells (PBMCs) isolated from 10 healthy donors as a control for the study. The third set comprised 91 tumors from head and neck cancer patients. These samples were used for IHC analysis for IL-12Rβ2 and to examine the correlation between IL-12Rβ2 expression level and cancer metastasis.

Quantitative RT-PCR.

Quantitative PCR was performed using the StepOnePlus real-time PCR system (Applied Biosystems Inc., Foster City, Calif.). The primer sequences used for real-time PCR are listed in following table of primer list.

Flow Cytometry.

Cells were harvested and washed twice with PBS. The cells were then incubated with primary antibodies (see following table of antibody list) for 1 hr at 4° C. and then with secondary antibodies for 30 min at 4° C. The stained cells were analyzed on a Cytomics™ FC500 Flow Cytometry apparatus (Beckman Coulter, Inc., Brea, Calif.) using Cytomics CXP Analysis software (Beckman Coulter, Inc., Brea, Calif.).

Western Blot.

These procedures were performed as previously described (Hsu et al., 2014). The results were measured using a GE LAS-4000 (GE Healthcare Inc., Marlborough, Mass.).

Macrophage Depletion.

Liposomal clodronate and phosphate buffer solution liposomes were purchased from ClodronateLiposomes.org (Haarlem, Netherlands). The concentration of clodronate in the liposome formulation was 5 mg/ml. A single dose of liposomal clodronate was administered via intraperitoneal (1 mg/mouse) or intratracheal (0.5 mg/mouse) injection at the indicated times.

Endothelial Cell Capillary Formation Assay.

Conditioned medium (obtained from BMDMs, sorted TAMs, PMDM, M1 macrophages, or M2 macrophages) was used to resuspend 5×10⁴ HUVECs, which were then seeded directly on Matrigel. After 12 hr, capillary formation was analyzed and quantified by measuring the number of branches.

Ingenuity Pathway Analysis.

Pathway and global functional analyses were performed using the Ingenuity Pathway Analysis (IPA; Ingenuity® Systems, www.ingenuity.com) as previously described (Hsu et al., 2014). Briefly, a dataset containing gene identifiers and corresponding expression values was uploaded, and each gene was mapped using the Ingenuity Pathways Knowledge Base (IPKB).

Analyses of Public Databases and GSEA.

Survival curves of gene expression in lung and gastric cancer patients were obtained from the website (http://kmplot.com/analysis/). GSEA was performed using the JAVA program (http://www.broadinstitute.org/gsea). The core EMT gene signatures (Taube, et al., 2010) were used to integrate the transcriptome changes in the M1- and M2-CM-treated A549 cells.

Preparation of Human Monocytes.

Peripheral mononuclear cells were isolated from the blood of healthy donors by standard density gradient centrifugation with Ficoll-Paque (Amersham Biosciences, Inc., Piscataway, N.J.). CD14+ cells were subsequently purified from peripheral mononuclear cells by high-gradient magnetic sorting using anti-CD14 microbeads (No. 130-050-201, Miltenyi Biotec GmbH, Bergisch Gladbach, Germany). The CD14⁺ monocytes were cultured in RPMI-1640 medium (Life Technologies, Inc., Gaithersburg, Md.) supplemented with hM-CSF for 5 days for the polarization of M0 macrophages. Fresh medium supplemented with hM-CSF (10 ng/ml) was added on day three.

Macrophage Polarization and Conditioned Media Collection.

The M0 macrophages were polarized into M1 or M2 macrophages by adding 1 μg/ml LPS plus 20 ng/ml IFN-γ or 20 ng/ml IL-4 plus 0.1 μM dexamethasone in 5% FBS RPMI-1460 medium, respectively. After 48 hr, the media of the polarized macrophages were changed into fresh media for another 48 hr, which served as the different M1- and M2-conditioned media.

Enzyme-Linked Immunosorbent Assay (ELISA)

Conditioned media were assayed using IL-35 ELISA kits (cat. no. 440508 and 439508 BioLegend, Inc.). Sorted TAMs or polarized macrophages were seeded and cultured for 24 hours. Supernatants were collected after centrifugation, and IL-35 was measured by ELISA.

Immunofluorescence.

The cells were seeded on poly-L-lysine-coated slides, fixed with 4% paraformaldehyde, and permeabilized with 0.5% Triton X-100. DAPI was used for nuclear staining. The images were captured using an Olympus FluoView FV10i laser scanning confocal microscope (Olympus Corporation, Tokyo, Japan) equipped with a 60× oil objective (Olympus UPLSAPO 60XO, NA 1.35). Images were collected sequentially using the confocal laser scanning microscope and analyzed using Olympus FV10-ASW Version 3.0 Software.

Immunohistochemistry.

Deparaffinization, rehydration, antigen retrieval (10 mM sodium citrate buffer, pH 6.0), permeabilization, antibody hybridization and visualization were performed as previously described (Yang et al., 2010). For immunohistochemical grading, the intensity of IL-12Rβ2 were defined as 0, 1+, 2+, or 3+. The immunoscore (H score) was defined by the intensity (0-3+) multiplied by the expression percentage (0-100) for each sample. The slides were independently scored by two individuals.

Cell Viability and Proliferation Assay.

For the cell viability assay, 1×10⁴ cells were seeded per well in a 96-well plate and incubated overnight and then treated with various concentrations of reagents. After 24 h, the growth medium was discarded, and MTT assay solution was added for 1 h at 37. Newly formed mitochondrial MTT crystals were dissolved with dimethyl sulfoxide, and the absorbance was read using a microplate reader.

Cell Migration Assays.

Cell migration was evaluated using Transwells with 8 μm filter membrane-containing upper chambers (Greiner Bio-One). Cells (2×10⁵) suspended in 100 μl of 0.5% FBS culture medium were applied to the upper chamber, and 600 μl of 10% FBS medium was added to the lower chamber. After 24 h, the membranes were fixed with 4% PFA and then stained for visualization.

Accession Numbers.

The datasets for the cDNA microarray for the conditioned media-treated A549 cells were deposited at the Gene Expression Omnibus (GEO) with the accession number GSE596943. The datasets for the cDNA microarray for the pTAMs vs. mTAMs (BMDM as a control) from the 4T1 mouse model were deposited at the Gene Expression Omnibus (GEO) with the accession number GSE596944.

Statistical Analysis.

A two-tailed independent Student's t-test was used to compare the continuous variables between the two groups. The chi-square test was applied to compare non-dichotomous variables. Kaplan-Meier estimation and log-rank test were used to compare survival between the patient groups. All statistical data were collected independently and analyzed by at least two independent experiments; p-values <0.05 were considered significant.

Table of antibodies used in the present invention:
Antibodies

| Protein | Application | Antibody | Origin | Incorporation |
|---|---|---|---|---|
| F4/80 | IHC | 14-4801 | rat | Thermo Fisher Scientific Inc. (Waltham, MA) |
| IL-12RB2 | IHC | ab198833 | rabbit pAb | Abcam Plc. (Cambridge, UK) |
| Vimentin | WB, IF | V6630 | mouse mAb | Sigma-Aldrich (St. Louis, MO) |
| N-cadherin | WB, IF | 610921 | mouse mAb | BD Transduction Laboratories ™ (Franklin Lakes, NJ) |
| E-cadherin | WB, IF | #4065 | rabbit pAb | Cell Signaling Technology, Inc. (Danvers, MA) |
| IL-35 | IF, neutralization | MAB-200-IL3522 | mouse mAb | Shenandoah Biotechnology, Inc. (Warwick, PA) |
| IL-12RB2 | WB, IF, FC | GTX103166 | rabbit pAb | GeneTex Inc. (Irvine, CA) |
| γ-catenin | WB | 610253 | mouse mAb | BD Transduction Laboratories ™ (Franklin Lakes, NJ) |
| Ebi3 subunit | WB | MAB18341 | rat mAb | R&D Systems, Inc. (Minneapolis, MN) |
| Il12a | WB | MAB1570 | mouse mAb | R&D Systems, Inc. (Minneapolis, MN) |
| β-actin | WB | MAB1501 | mouse mAb | Chemicon International Inc. (Temecula, CA) |
| CSF-1R | neutralization | 135504 | rat | BioLegend, Inc. (San Diego, CA) |
| CD11b-PE | FC | 130-091-240 | rat | Miltenyi Biotec GmbH (Teterow, Germany) |
| F4/80-APC | FC | 123116 | rat | BioLegend, Inc. (San Diego, CA) |
| Ly6C-FITC | FC | 130-102-295 | rat | Miltenyi Biotec GmbH (Teterow, Germany) |
| HLADR-FITC | FC | SAB4700659 | mouse mAb | |
| CD163 | FC | MAC1853 | mouse mAb | Bio-Rad Laboratories, Inc. (Hercules, CA) |
| mannose receptor | FC | 321102 | mouse mAb | BioLegend, Inc. (San Diego, CA) |
| (MR) | | | | |

Table of primers used in the present invention:

| SEQ | | | | SEQ | | | |
|---|---|---|---|---|---|---|---|
| ID NO: | Gene name | | Sequence 5′-3′ | ID NO: | Gene name | | Sequence 5′-3′ |
| 01 | TNF | F | TCAGCCTCTTCTCCTTCCTG | 25 | IL6 | F | GTCAGGGGTGGTTATTGCAT |
| 02 | TNF | R | GCCAGAGGGCTGATTAGAGA | 26 | IL6 | R | AGTGAGGAACAAGCCAGAGC |
| 03 | IL1B | F | AAGCCCTTGCTGTAGTGGTG | 27 | IL10 | F | TCAAACTCACTCATGGCTTTGT |
| 04 | IL1B | R | GAAGCTGATGGCCCTAAACA | 28 | IL10 | R | GCTGTCATCGATTTCTTCCC |
| 05 | CD163 | F | TGAGCCACACTGAAAAGGAA | 29 | CCL18 | F | GTGGAATCTGCCAGGAGGTA |
| 06 | CD163 | R | GGTGAATTTCTGCTCCATTCA | 30 | CCL18 | R | TCCTTGTCCTCGTCTGCAC |
| 07 | MRC1 | F | CAGCGCTTGTGATCTTCATT | 31 | IL12A | F | TTCACCACTCCCAAAACCTGC |
| 08 | MRC1 | R | TACCCCTGCTCCTGGTTTTT | 32 | IL12A | R | GAGGCCAGGCAACTCCCATTAG |
| 09 | EB13 | F | CAGCTTCGTGCCTTTCATAA | 33 | IL12RB2 | F | AGACCTCAGTGGTGTAGCAGAG |
| 10 | EB13 | R | CTCCCACTGCACCTGTAGC | 34 | IL12RB2 | R | TGATGACCAGCGGTTCAGGATC |
| 11 | Nos2 | F | GTCGATGTCACATGCAGCTT | 35 | Tnf | F | GGTCTGGGCCATAGAACTGA |
| 12 | Nos2 | R | GAAGAAAACCCCTTGTGCTG | 36 | Tnf | R | CAGCCTCTTCTCATTCCTGC |
| 13 | II15 | F | CTGCCATCCATCCAGAACTC | 37 | Cxcl9 | F | TAGGCAGGTTTGATCTCCGT |
| 14 | II15 | R | AGCACTGCCTCTTCATGGTC | 38 | Cxcl9 | R | CGATCCACTACAAATCCCTCA |
| 15 | Cxcl10 | F | CCTATGGCCCTCATTCTCAC | 39 | Arg1 | F | TTTTTCCAGCAGACCAGCTT |
| 16 | Cxcl10 | R | CTCATCCTGCTGGGTCTGAG | 40 | Arg1 | R | AGAGATTATCGGAGCGCCTT |
| 17 | Mrc1 | F | GTGGATTGTCTTGTGGAGCA | 41 | Il10 | F | AGACACCTTGGTCTTGGAGC |
| 18 | Mrc1 | R | TTGTGGTGAGCTGAAAGGTG | 42 | Il10 | R | TTTGAATTCCCTGGGTGAGA |
| 19 | Chil3 | F | TTTCTCCAGTGTAGCCATCCTT | 43 | Ccl17 | F | ACCAGCTCACCAACTTCCTG |
| 20 | Chil3 | R | AGGAGCAGGAATCATTGACG | 44 | Ccl17 | R | TGCTTCTGGGGACTTTTCTG |
| 21 | Il12a | F | TCTCCCACAGGAGGTTTCTG | 45 | Ebi3 | F | AGCGGAGTCGGTACTTGAGA |
| 22 | Il12a | R | ACAGAGTTCCAGGCCATCAA | 46 | Ebi3 | R | TCCTAGCCTTTGTGGCTGAG |
| 23 | Il12b2 | F | TGTGGGGTGGAGATCTCAGT | — | — | — | — |
| 24 | Il12b2 | R | TCTCCTTCCTGGACACATGA | — | — | — | — |

What is claimed is:

1. A method for treating cancer metastasis, comprising: administering a subject in need a therapeutically effective amount of an anti-interleukin-35 (IL-35) antibody or antigen-binding fragment thereof or a shRNA against the expression of IL12RB2;
wherein said cancer is breast cancer, non-small cell lung cancer, gastric cancer, head and neck cancer, colon cancer, pancreatic cancer, ovarian cancer, or oral cancer.

2. The method of claim 1, further comprising administering said subject a therapeutically effective amount of an anti-colony stimulating factor 1 receptor (CSF1R) antibody or antigen-binding fragment thereof.

3. The method of claim 2, wherein said anti-CSF1R antibody or antigen-binding fragment thereof is administered simultaneously or at any interval with said administering of said anti-IL-35 antibody or antigen-binding fragment thereof or said shRNA against the expression of IL12RB2.

4. The method of claim 1, wherein said subject had a primary cancer treatment before said administering.

5. The method of claim 4, wherein said primary cancer treatment is endocrine therapy, chemotherapy, radiotherapy, hormone therapy, surgery, gene therapy, thermal therapy, ultrasound therapy, or a combination thereof.

6. The method of claim 1, wherein said anti-IL-35 antibody is a humanized antibody.

7. The method of claim 2, wherein said anti-CSF1R antibody is a humanized antibody.

8. The method of claim 1, wherein said administering of said anti-IL-35 antibody or antigen-binding fragment thereof or said shRNA against the expression of IL12RB2 is conducted through intramuscular, intraperitoneal, intracerebrospinal, subcutaneous, intra-articular, intrasynovial, intrathecal, oral, inhalation or topical routes.

9. The method of claim 2, wherein said administering of said anti-CSF1R antibody or antigen-binding fragment thereof is conducted through intramuscular, intraperitoneal, intracerebrospinal, subcutaneous, intra-articular, intrasynovial, intrathecal, oral, inhalation or topical route.

10. The method of claim 1, wherein said anti-IL-35 antibody is administered with a pharmaceutically acceptable carrier.

11. The method of claim 2, wherein said anti-CSF1R antibody is administered with a pharmaceutically acceptable carrier.

12. The method of claim 1, wherein said therapeutically effective amount of said anti-IL-35 antibody or antigen-binding fragment thereof or said shRNA against the expression of IL2RB2 is 0.01 to 20 mg/kg body weight of said subject.

13. The method of claim 2, wherein said therapeutically effective amount of said anti-CSF1R antibody or antigen-binding fragment thereof is 0.01 to 20 mg/kg body weight of said subject.

14. A method for treating cancer, comprising:
(a) administering a subject in need with a primary cancer treatment; and
(b) administering said subject a therapeutically effective amount of an anti-IL-35 antibody or antigen-binding fragment thereof or a shRNA against the expression of IL12RB2 and a therapeutically effective amount of an anti-CSF1R antibody or antigen-binding fragment thereof;
wherein said cancer is breast cancer, non-small cell lung cancer, gastric cancer, head and neck cancer, colon cancer, pancreatic cancer, ovarian cancer, or oral cancer.

15. The method of claim 14, wherein said primary cancer treatment is endocrine therapy, chemotherapy, radiotherapy, hormone therapy, surgery, gene therapy, thermal therapy, ultrasound therapy, or a combination thereof.

16. The method of claim 14, wherein said anti-IL-35 antibody is a humanized antibody and/or said anti-CSF1R antibody is a humanized antibody.

17. The method of claim 14, wherein said administering of said anti-IL-35 antibody or antigen-binding fragment thereof or said shRNA against the expression of IL12RB2 is conducted through intramuscular, intraperitoneal, intracerebrospinal, subcutaneous, intra-articular, intrasynovial, intrathecal, oral, inhalation or topical route.

18. The method of claim 14, wherein said administering of said anti-CSF1R antibody or antigen-binding fragment thereof is conducted through intramuscular, intraperitoneal, intracerebrospinal, subcutaneous, intra-articular, intrasynovial, intrathecal, oral, inhalation or topical routes.

19. The method of claim 14, wherein said therapeutically effective amount of said anti-IL-35 antibody or antigen-binding fragment thereof or said shRNA against the expression of IL12RB2 is 0.01 to 20 mg/kg body weight of said subject.

20. The method of claim 14, wherein said therapeutically effective amount of said anti-CSF1R antibody or antigen-binding fragment thereof is 0.01 to 20 mg/kg body weight of said subject.
